(19) United States
(12) Patent Application Publication
(10) Pub. No.: US 2024/0369536 A1
(43) Pub. Date: Nov. 7, 2024

(54) A CELL BASED TEST TO MONITOR DMARD DRUG RESPONSE

(71) Applicant: UNIVERSITY OF ULSTER, Londonderry (GB)

(72) Inventors: Tony Bjourson, Londonderry (GB); David Gibson, Londonderry (GB)

(73) Assignee: UNIVERSITY OF ULSTER, Londonderry (GB)

(21) Appl. No.: 18/572,349

(22) PCT Filed: Jun. 24, 2022
(86) PCT No.: PCT/EP2022/067438

§ 371 Date: Dec. 20, 2023

(30) Foreign Application Priority Data
Jun. 25, 2021 (GB) 2109179.8

(51) Int. Cl. G01N 33/50

(52) U.S. Cl. CPC G01N 33/505; G01N 33/5023

(57) ABSTRACT

This invention relates to predicting a subject's responsiveness to Disease Modifying Anti-Rheumatic Drug therapy of rheumatoid arthritis. The invention provides an in vitro method for predicting responsiveness to a Disease Modifying Anti-Rheumatic Drug therapy in a subject, the method comprising the steps of: (a) providing a biological sample; (b) detecting the presence, absence, or quantitative level of a first marker in the biological sample, wherein the first marker is a regulatory T cell having a CD45RA+FoxP3− phenotype; (c) correlating the presence, absence, or quantitative level of the first marker to the predicted responsiveness to the Disease Modifying Anti-Rheumatic Drug therapy in the subject.

### FIELD OF THE INVENTION

The present invention relates to predicting a subject's responsiveness to Disease Modifying Anti-Rheumatic Drug therapy of rheumatoid arthritis.

### BACKGROUND OF THE INVENTION

Rheumatoid arthritis (RA) is a chronic condition characterised by an autoimmune response targeted primarily to synovial joints. Tenderness and swelling of joints are typical symptoms of the disease, as well as elevated blood-based inflammatory markers C-reactive protein (CRP) and erythrocyte sedimentation rate (ESR). These outcome measures are combined into a composite disease activity score of 28 joints (DAS28), which incorporates the number of swollen and tender joints, the concentration of ESR or CRP, and the general health of the patient. The DAS28 measurement/level can be used along with the patient's rating of their general health on a 0-10 scale. DAS28 allows clinicians to monitor disease activity and assists in determining treatment response. However, composite disease activity scores such as the DAS28 may be unreliable. The ESR and CRP components of DAS28 are non-specific markers of RA disease activity. The visual analogue scale (VAS) and tender joint count components of DAS 28 can also be subjective, further emphasizing the need for more accurate and reliable measures of disease activity.

Disease activity in RA stems from activation of immune cells found in peripheral blood and affected synovial tissue. Once activated, these immune cells can directly or indirectly promote secretion of degradative enzymes within the synovial joint, increasing cartilage breakdown and bone erosion. T cells play a pivotal role in moderating the adaptive immune response in healthy individuals. Specifically, regulatory T cells (Tregs) reduce the actions of damage-causing effector T cells (Teffs). However, in the RA autoimmune environs, Treg suppression of Teffs is less effective. Therefore, secretion of cytokines and recruitment of other immune cells to the synovial joint by Teffs is unchecked, thus increasing the risk of irreversible damage.

CD4+CD25+ Treg density is increased in the synovial fluid of RA patients compared to peripheral blood, and overall Treg numbers are elevated in RA compared to healthy individuals. Furthermore, synovial fluid Tregs exhibit elevated activation markers including transcription factor Forkhead box P3 (FoxP3) and cytotoxic T lymphocyte protein-4 (CTLA-4). A recent study observed increased CD4+CD25+FoxP3+ Tregs with a lower activation state in RA patients, as measured by activation markers CD69 and CD71. However, the association with individual responses to treatment were unclear. When patients were classified according to anti-TNF response, increased numbers of CD4+CD25+ Tregs were observed in peripheral blood of responders relative to non-responders. This finding was also associated with reduced CRP levels in responders, suggesting peripheral Treg numbers may be associated with disease activity and drug response in RA. However, further evidence is needed to clearly define changes in relative numbers of peripheral Tregs, their specific immunophenotypes and activation levels in both treatment response and non-response.

Treg activity during inflammatory episodes is influenced by interaction with monocytes and macrophages. In vitro studies have demonstrated the ability of synovial monocytes to promote Th1 and Th17 responses in CD4+ T cells. For example, intracellular levels of inflammatory cytokines IL-17, IFN-γ, TNFα and IL-10 increase in Tregs as a result of exposure to activated monocytes. Furthermore, increased IL-6 and IL-23 secretion from monocyte derived dendritic cells, has been associated with the induction of Th17 cells and Tregs in RA.

Monocyte phenotypes may also represent potential markers of disease activity in RA. Sialic acid-binding immunoglobulin-like lectin 1 (Siglec-1, also known as CD169), which is an adhesion molecule restricted to monocytes and macrophages, is elevated both intracellularly and at the cell surface in line with increases in DAS28 and CRP. Furthermore, Siglec-1 levels subside along with disease activity improvements in response to treatment. The mechanism behind the Siglec-1 elevation in RA is not fully understood, however it may be implicated in pro-inflammatory pathways, resulting in interferon-gamma (IFN-γ) secretion from activated T cells. Furthermore, Siglec-1 binds to ligands on the surface of Tregs, an action that may contribute to Treg modulation in RA. CD43, a cell-surface sialoglycoprotein, is one such Treg based Siglec-1 ligand that has previously been associated with in vitro Treg activation. However, regarding study of the interaction between Tregs and monocytes via CD43 and Siglec-1 has not been made.

Increased Siglec-1 expression on CD14+CD16+ resident monocytes as well as CD14+CD16− classical monocytes of RA patients compared to healthy controls has also been described. Resident monocytes can be further subdivided into CD14highCD16+ intermediate and CD14lowCD16+ non-classical monocytes. Several studies highlight the importance of considering these three monocyte subsets in RA. Synovial CD16+ monocytes increase in RA, whereas peripheral blood CD16− monocytes are reduced in RA compared to healthy controls. Furthermore, several studies showcase the potential for baseline monocyte subsets to predict treatment responses in RA to anti-TNF therapy and methotrexate (MTX), where relative numbers of classical and intermediate monocytes increase in non-responders compared to responders. However, further evidence is needed to fully understand the balance between monocytes subsets and disease activity measures in RA, specifically in relation to Siglec-1 positive subpopulations.

Treat to target is a treatment strategy for RA that broadly involves regular testing to monitor the success of the current treatment regimen, and promptly switching treatment regimen if treatment progress is not made.

Current treat to target strategies for RA employing DMARDs usually require 3-6 months to tell if a particular treatment is effective in reducing disease activity into a state of remission; and it can take two to three cycles of various combinations of treatments to reach the ideal drug and dose. The lengthy time required to determine the optimal treatment regimen leads to extended periods of poorly controlled disease. Elevated disease activity increases the risk of irreversible joint damage, disability and inability to work or perform every day tasks. Reducing the time taken to reach an optimal treatment regimen would have several advantages, including: reducing the patient's exposure to ineffective treatment and the side effects thereof; reducing the duration of poorly controlled disease and so the risks of irreversible joint damage; allowing an earlier start to optimal treatment leading to a quicker and/or improved treatment outcome; reducing the costs of ineffective treatment and allowing more efficient prioritisation of treatment resources to appropriate patients, allowing for more effective use of treatment resources.

Therefore, there is a need for an improved means to determine or predict response to a treatment regiment at an earlier stage of treatment. For example, an improved test for predicting response to a DMARD treatment in an RA patient, or an improved method for determining response to DMARD treatment in an RA patient.

### SUMMARY OF THE INVENTION

According to a first aspect of the present invention, there is provided a method for predicting responsiveness to a Disease Modifying Anti-Rheumatic Drug therapy in a subject, the method comprising the steps of:
b) detecting the presence, absence, or quantitative level of a first marker in a biological sample;
c) correlating the presence, absence, or quantitative level of the first marker to the predicted responsiveness to the Disease Modifying Anti-Rheumatic Drug therapy in the subject.

Optionally, there is provided a method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample;
c) correlating the presence, absence, or quantitative level of the first marker to the predicted responsiveness to the Disease Modifying Anti-Rheumatic Drug therapy in the subject.

Optionally, the Disease Modifying Anti-Rheumatic Drug therapy in the subject is for therapy of rheumatoid arthritis; such that the method is for predicting responsiveness to a Disease Modifying Anti-Rheumatic Drug therapy of rheumatoid arthritis in a subject.

Optionally, the correlating step (c) comprises:
c) correlating the presence, absence, or quantitative level of the first marker to the predicted responsiveness to the Disease Modifying Anti-Rheumatic Drug therapy of rheumatoid arthritis in the subject.

Optionally, there is provided a method for predicting responsiveness to a Disease Modifying Anti-Rheumatic Drug therapy of rheumatoid arthritis in a subject, the method comprising the steps of:
b) detecting the presence, absence, or quantitative level of a first marker in a biological sample;
c) correlating the presence, absence, or quantitative level of the first marker to the predicted responsiveness to the Disease Modifying Anti-Rheumatic Drug therapy of rheumatoid arthritis in the subject.

Optionally, there is provided a method for predicting responsiveness to a Disease Modifying Anti-Rheumatic Drug therapy of rheumatoid arthritis in a subject, the method comprising the steps of:
a) providing a biological sample;
b) detecting the presence, absence, or quantitative level of a first marker in the biological sample;
c) correlating the presence, absence, or quantitative level of the first marker to the predicted responsiveness to the Disease Modifying Anti-Rheumatic Drug therapy of rheumatoid arthritis in the subject.

Optionally, the method is an in vitro method. Optionally, the method is carried out in vitro.

Optionally, the correlating step (c) comprises:
c) correlating the quantitative level of the first marker to the predicted responsiveness to the Disease Modifying Anti-Rheumatic Drug therapy in the subject.

Optionally, the providing step (a) comprises:
a) providing an in vitro biological sample.

Optionally, the providing step (a) is carried out in vitro.

Optionally, the detecting step (b) is carried out in vitro.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample;
c) correlating the presence, absence, or quantitative level of the first marker to the predicted responsiveness to the Disease Modifying Anti-Rheumatic Drug therapy in the subject.

Optionally, the detecting step (b) comprises:
b) detecting the quantitative level of a first marker in the biological sample.

Optionally, the correlating step (c) comprises:
c) correlating the quantitative level of the first marker to the predicted responsiveness to the Disease Modifying Anti-Rheumatic Drug therapy in the subject.

Optionally, the method further comprises a stimulating step (a1) wherein providing step (a) precedes stimulating step (a1), and wherein stimulating step (a1) precedes detecting step (b), wherein stimulating step (a1) comprises:
a1) stimulating the biological sample with the Disease Modifying Anti-Rheumatic Drug.

Optionally, the biological sample comprises a culture, optionally an in vitro culture.

Optionally, the culture comprises a blood culture. Optionally, the culture is a blood culture.

Optionally, the first marker is a regulatory T cell having a CD45RA⁺FoxP3⁻ phenotype.

Optionally, the first marker is a cell having a CD4⁺CD25⁺CD127⁻CD45RA⁺FoxP3⁻ phenotype. Optionally, the method comprises the steps of:
a) providing a biological sample;
b) detecting the presence, absence, or quantitative level of a first marker in the biological sample; and detecting the presence, absence, or quantitative level of a second marker in the biological sample;
c) correlating the presence, absence, or quantitative level of the first marker and the presence, absence, or quantitative level of the second marker; to the predicted responsiveness to the Disease Modifying Anti-Rheumatic Drug therapy in the subject.

Optionally, the detecting step (b) comprises:
c) detecting the quantitative level of a first marker in the biological sample; and detecting the quantitative level of a second marker in the biological sample.

Optionally, the correlating step (c) comprises:
c) correlating the quantitative level of the first marker and the quantitative level of the second marker, to the predicted responsiveness to the Disease Modifying Anti-Rheumatic Drug therapy in the subject.

Optionally, the second marker is a regulatory T cell having a CD45RA⁻FoxP3⁺ phenotype.

Optionally, the second marker is a cell having a CD4⁺CD25⁺CD127⁻CD45RA⁻FoxP3⁻ phenotype Optionally, the detecting step (b) further comprises:
b) detecting the presence, absence or quantitative level of a third marker in the biological sample.

Optionally, the detecting step (b) further comprises:
b) detecting the quantitative level of third marker in the biological sample.

Optionally, the third marker is a regulatory T cell.

Optionally, the third marker is a cell having a CD4⁺CD25⁺CD127⁻ phenotype.

Optionally, the quantitative level of the first marker is a relative level of the first marker as a proportion of the quantitative level of the third marker.

Optionally, the quantitative level of the second marker is a relative level of the second marker as a proportion of the quantitative level of the third marker.

Optionally, a relative level of the first marker greater than a threshold proportion correlates to a predicted low responsiveness to the Disease Modifying Anti-Rheumatic Drug therapy in the subject.

Optionally, a relative level of the first marker less than the threshold proportion correlates to a predicted high responsiveness to the Disease Modifying Anti-Rheumatic Drug therapy in the subject.

Optionally, a relative level of the second marker less than the threshold proportion correlates to a predicted low responsiveness to the Disease Modifying Anti-Rheumatic Drug therapy in the subject.

Optionally, a relative level of the second marker greater than the threshold proportion correlates to a predicted high responsiveness to the Disease Modifying Anti-Rheumatic Drug therapy in the subject.

Optionally, a relative level of the first marker greater than a threshold proportion of the quantitative level of the third marker correlates to a predicted low responsiveness to the Disease Modifying Anti-Rheumatic Drug therapy in the subject. Optionally, a relative level of the first marker less than the threshold proportion of the quantitative level of the third marker correlates to a predicted high responsiveness to the Disease Modifying Anti-Rheumatic Drug therapy in the subject. Optionally, a relative level of the second marker less than the threshold proportion of the quantitative level of the third marker correlates to a predicted low responsiveness to the Disease Modifying Anti-Rheumatic Drug therapy in the subject. Optionally, a relative level of the second marker greater than the threshold proportion of the quantitative level of the third marker correlates to a predicted high responsiveness to the Disease Modifying Anti-Rheumatic Drug therapy in the subject.

Optionally, the threshold proportion is selected from the group comprising: 1%, 5%, 10%, 11%, 12%, 13%, 14%, 15%, 16%, 17%, 18%, 19%, 19.5%, 19.9%, 20%, 21%, 21.5%, 21.6%, 21.7%, 22%, 23%, 24%, 25%, 26%, 27%, 28%, 29%, 30%, 35%, 40%, 50%, 60%, 70%, 80%, and 90%.

Optionally, a relative level of the first marker greater than 20% correlates to a predicted low responsiveness to the Disease Modifying Anti-Rheumatic Drug therapy in the subject. Optionally, a relative level of the first marker greater than 20% of the quantitative level of the third marker correlates to a predicted low responsiveness to the Disease Modifying Anti-Rheumatic Drug therapy in the subject.

Optionally, a relative level of the first marker less than 20% correlates to a predicted high responsiveness to the Disease Modifying Anti-Rheumatic Drug therapy in the subject. Optionally, a relative level of the first marker less than 20% of the quantitative level of the third marker correlates to a predicted high responsiveness to the Disease Modifying Anti-Rheumatic Drug therapy in the subject.

Optionally, a relative level of the first marker greater than 19.9% correlates to a predicted low responsiveness to the Disease Modifying Anti-Rheumatic Drug therapy in the subject. Optionally, a relative level of the first marker greater than 19.9% of the quantitative level of the third marker correlates to a predicted low responsiveness to the Disease Modifying Anti-Rheumatic Drug therapy in the subject.

Optionally, a relative level of the first marker less than 19.9% correlates to a predicted high responsiveness to the Disease Modifying Anti-Rheumatic Drug therapy in the subject. Optionally, a relative level of the first marker less than 19.9% of the quantitative level of the third marker correlates to a predicted high responsiveness to the Disease Modifying Anti-Rheumatic Drug therapy in the subject.

Optionally, a relative level of the second marker less than 25% correlates to a predicted low responsiveness to the Disease Modifying Anti-Rheumatic Drug therapy in the subject. Optionally, a relative level of the second marker less than 25% of the quantitative level of the third marker correlates to a predicted low responsiveness to the Disease Modifying Anti-Rheumatic Drug therapy in the subject.

Optionally, a relative level of the second marker greater than 25% correlates to a predicted high responsiveness to the Disease Modifying Anti-Rheumatic Drug therapy in the subject. Optionally, a relative level of the second marker greater than 25% of the quantitative level of the third marker correlates to a predicted high responsiveness to the Disease Modifying Anti-Rheumatic Drug therapy in the subject.

Optionally, a relative level of the second marker less than 26% correlates to a predicted low responsiveness to the Disease Modifying Anti-Rheumatic Drug therapy in the subject. Optionally, a relative level of the second marker less than 26% of the quantitative level of the third marker correlates to a predicted low responsiveness to the Disease Modifying Anti-Rheumatic Drug therapy in the subject.

Optionally, a relative level of the second marker greater than 26% correlates to a predicted high responsiveness to the Disease Modifying Anti-Rheumatic Drug therapy in the subject. Optionally, a relative level of the second marker greater than 26% of the quantitative level of the third marker correlates to a predicted high responsiveness to the Disease Modifying Anti-Rheumatic Drug therapy in the subject.

Optionally, a relative level of the second marker less than 22% correlates to a predicted low responsiveness to the Disease Modifying Anti-Rheumatic Drug therapy in the subject. Optionally, a relative level of the second marker less than 22% of the quantitative level of the third marker correlates to a predicted low responsiveness to the Disease Modifying Anti-Rheumatic Drug therapy in the subject.

Optionally, a relative level of the second marker greater than 22% correlates to a predicted high responsiveness to the Disease Modifying Anti-Rheumatic Drug therapy in the subject. Optionally, a relative level of the second marker greater than 22% of the quantitative level of the third marker correlates to a predicted high responsiveness to the Disease Modifying Anti-Rheumatic Drug therapy in the subject.

Optionally, a relative level of the second marker less than 21.6% correlates to a predicted low responsiveness to the Disease Modifying Anti-Rheumatic Drug therapy in the subject. Optionally, a relative level of the second marker less than 21.6% of the quantitative level of the third marker correlates to a predicted low responsiveness to the Disease Modifying Anti-Rheumatic Drug therapy in the subject.

Optionally, a relative level of the second marker greater than 21.6% correlates to a predicted high responsiveness to the Disease Modifying Anti-Rheumatic Drug therapy in the subject. Optionally, a relative level of the second marker greater than 21.6% of the quantitative level of the third marker correlates to a predicted high responsiveness to the Disease Modifying Anti-Rheumatic Drug therapy in the subject.

Optionally, the Disease Modifying Anti-Rheumatic Drug is selected from the group comprising: abatacept, adalimumab, anakinra, azathioprine, chloroquine, ciclosporin, D-penicillamine, etanercept, golimumab, gold salts, hydroxychloroquine, infliximab, leflunomide, methotrexate, minocycline, rituximab, sulfasalazine, tocilizumab, and tofacitinib.

Preferably, the Disease Modifying Anti-Rheumatic Drug is selected from the group comprising: hydroxychloroquine, leflunomide, methotrexate, and sulfasalazine.

Optionally, the biological sample substantially comprises bodily fluid. Optionally, the biological sample comprises bodily fluid. Optionally, the biological sample consists of bodily fluid. Optionally, the biological sample is a bodily fluid.

Optionally, the biological sample substantially comprises blood. Optionally, the biological sample comprises blood. Optionally, the biological sample consists of blood. Optionally, the biological sample is blood.

Optionally, the biological sample substantially comprises a blood sample. Optionally, the biological sample comprises a blood sample. Optionally, the biological sample consists of a blood sample.

Optionally, the biological sample is a blood sample.

Optionally, the biological sample is from a single organism. Optionally, the biological sample is from a single subject. Optionally, the biological sample is from a single patient.

Optionally, the biological sample is from several organisms. Optionally, the biological sample is from several subjects. Optionally, the biological sample is from several patients.

Optionally, the biological sample is from the subject.

Optionally, the biological sample is from the subject; wherein the biological sample represents the subject in receipt of Disease Modifying Anti-Rheumatic Drug therapy. Optionally, the biological sample is from the subject; wherein the biological sample represents the subject in receipt of Disease Modifying Anti-Rheumatic Drug therapy in the subject.

Optionally, the detecting step (b) comprises antibody labelling of at least one marker selected from the group comprising: the first marker, the second marker, the third marker, and combinations thereof.

Optionally, the detecting step (b) comprises antibody labelling of at least one marker selected from the group comprising: a regulatory T cell having a CD45RA+FoxP3− phenotype, a regulatory T cell having a CD45RA−FoxP3+ phenotype, a regulatory T cell, and combinations thereof.

Optionally, the detecting step (b) comprises antibody labelling of at least one marker selected from the group comprising: a regulatory T cell having a CD45RA+FoxP3− phenotype, a regulatory T cell having a CD45RA−FoxP3+ phenotype, a regulatory T cell, and combinations thereof.

Optionally, the detecting step (b) comprises antibody labelling of at least one marker selected from the group comprising: a cell having a CD4+CD25+CD127−CD45RA+FoxP3− phenotype, a cell having a CD4+CD25+CD127−CD45RA+FoxP3− phenotype, a cell having a CD4+CD25+CD127− phenotype, and combinations thereof.

Optionally, the detecting step (b) comprises flow cytometry, optionally fluorescence-activated cell sorting.

Optionally, the detecting step (b) comprises microscopy.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a regulatory T cell having a CD45RA⁺FoxP3⁻ phenotype;
c) correlating the presence, absence, or quantitative level of the first marker to the predicted responsiveness to the Disease Modifying Anti-Rheumatic Drug therapy in the subject.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a regulatory T cell having a CD45RA⁺FoxP3⁻ phenotype; and detecting the presence, absence, or quantitative level of a second marker in the biological sample, wherein the second marker is a regulatory T cell having a CD45RA⁻FoxP3⁺ phenotype;
c) correlating the presence, absence, or quantitative level of the first marker and the presence, absence, or quantitative level of the second marker; to the predicted responsiveness to the Disease Modifying Anti-Rheumatic Drug therapy in the subject.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a regulatory T cell having a CD45RA⁺FoxP3⁻ phenotype; and detecting the presence, absence, or quantitative level of a second marker in the biological sample, wherein the second marker is a regulatory T cell having a CD45RA⁻FoxP3⁺ phenotype; and detecting the presence, absence or quantitative level of a third marker in the biological sample, wherein the third marker is a regulatory T cell;
c) correlating the presence, absence, or quantitative level of the first marker and the presence, absence, or quantitative level of the second marker; to the predicted responsiveness to the Disease Modifying Anti-Rheumatic Drug therapy in the subject.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a cell having a CD4⁺CD25⁺CD127⁻ CD45RA⁺FoxP3⁻ phenotype;
c) correlating the presence, absence, or quantitative level of the first marker to the predicted responsiveness to the Disease Modifying Anti-Rheumatic Drug therapy in the subject.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a cell having a CD4⁺CD25⁺CD127⁻ CD45RA⁺FoxP3⁻ phenotype; and detecting the presence, absence, or quantitative level of a second marker in the biological sample, wherein the second marker is a cell having a CD4⁺CD25⁺CD127⁻CD45RA⁻FoxP3⁺ phenotype;
c) correlating the presence, absence, or quantitative level of the first marker and the presence, absence, or quantitative level of the second marker; to the predicted responsiveness to the Disease Modifying Anti-Rheumatic Drug therapy in the subject.

Optionally, there is provided an in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a cell having a CD4⁺CD25⁺CD127⁻ CD45RA⁺FoxP3⁻ phenotype; and detecting the presence, absence, or quantitative level of a second marker in the biological sample, wherein the second marker is a cell having a CD4⁺CD25⁺CD127⁻CD45RA⁻FoxP3⁺ phenotype; and detecting the presence, absence or quantitative level of a third marker in the biological sample, wherein the third marker is a cell having a CD4⁺CD25⁺CD127⁻ phenotype;
c) correlating the presence, absence, or quantitative level of the first marker and the presence, absence, or quantitative level of the second marker; to the predicted responsiveness to the Disease Modifying Anti-Rheumatic Drug therapy in the subject.

Optionally, there is provided a method for testing predicted responsiveness to a Disease Modifying Anti-Rheumatic Drug therapy in a subject; the method comprising the steps of
(i) providing a culture;
(ii) stimulating the culture with the Disease Modifying Anti-Rheumatic Drug;
(iii) detecting the presence, absence, or quantitative level of a first marker in the culture;
(iv) correlating the presence, absence, or quantitative level of the first marker to the predicted responsiveness to the Disease Modifying Anti-Rheumatic Drug therapy in the subject.

Optionally, there method is an in vitro method.

Optionally, the culture is an in vitro culture.

Optionally, there is provided an in vitro method for testing predicted responsiveness to a Disease Modifying Anti-Rheumatic Drug therapy in a subject; the method comprising the steps of
(i) providing a culture;
(ii) stimulating the culture with the Disease Modifying Anti-Rheumatic Drug;
(iii) detecting the presence, absence, or quantitative level of a first marker in the culture;
(iv) correlating the presence, absence, or quantitative level of the first marker to the predicted responsiveness to the Disease Modifying Anti-Rheumatic Drug therapy in the subject.

Optionally, the detecting step (ii) further comprises:
(ii) detecting the presence, absence, or quantitative level of a second marker in the biological sample.

Optionally, the detecting step (ii) further comprises:
(ii) detecting the presence, absence, or quantitative level of a third marker in the biological sample.

Optionally, the culture comprises a blood culture. Optionally, the culture is a blood culture.

Optionally, the first marker is a regulatory T cell having a CD45RA⁻FoxP3⁻ phenotype. Optionally, the first marker is a cell having a CD4⁺CD25⁺CD127⁻CD45RA⁺FoxP3⁻ phenotype.

Optionally, the second marker is a regulatory T cell having a CD45RA⁻FoxP3⁺ phenotype. Optionally, the second marker is a cell having a CD4⁺CD25⁺CD127⁻CD45RA⁻FoxP3⁺ phenotype.

Optionally, the third marker is a regulatory T cell. Optionally, the third marker is a cell having a CD4⁺CD25⁺CD127⁻ phenotype.

According to a second aspect of the present invention, there is provided a Disease Modifying Anti-Rheumatic Drug therapy response prediction kit, comprising at least one antibody against CD45RA and at least one antibody against FoxP3; optionally further comprising instructions for use.

Optionally, the kit further comprises at least one antibody selected from the group comprising: an antibody against CD4, an antibody against CD25, an antibody against CD127, and combinations thereof.

Optionally, the kit comprises at least one labelled antibody against CD45RA and at least one labelled antibody against FoxP3; optionally further comprising instructions for use.

Optionally, the kit further comprises at least one labelled antibody selected from the group comprising: a labelled antibody against CD4, a labelled antibody against CD25, a labelled antibody against CD127, and combinations thereof.

Optionally, the kit comprises labelled antibodies. Optionally, the antibody against CD45RA is a labelled antibody against CD45RA. Optionally, the antibody against FoxP3 is a labelled antibody against FoxP3. Optionally, the antibody against CD4 is a labelled antibody against CD4. Optionally, the antibody against CD25 is a labelled antibody against CD25. Optionally, the antibody against CD127 is a labelled antibody against CD127.

Optionally, the at least one labelled antibody is labelled with biotin.

Optionally, the at least one labelled antibody is labelled with an enzyme reporter. Optionally, the at least one labelled antibody is labelled with an enzyme reporter selected from the group comprising: horseradish peroxidase, alkaline phosphatase, glucose oxidase, and P-galactosidase.

Optionally, the at least one labelled antibody is fluorescently labelled. Optionally, the at least one labelled antibody is labelled with a fluorescent dye.

According to a third aspect of the present invention, there is provided use of a kit for predicting responsiveness to a Disease Modifying Anti-Rheumatic Drug therapy in a subject; wherein the kit comprises at least one antibody against CD45RA and at least one antibody against FoxP3; wherein the kit optionally further comprises instructions for use.

Optionally, the kit is the kit of the second aspect of the present invention; such that the use comprises use of the kit of the second aspect of the present invention.

Optionally, there is provided use of a kit for predicting responsiveness to a Disease Modifying Anti-Rheumatic Drug therapy in a subject; wherein the kit comprises at least one labelled antibody against CD45RA and at least one labelled antibody against FoxP3; wherein the kit optionally further comprises instructions for use.

Optionally, there is provided use of a kit for predicting responsiveness to a Disease Modifying Anti-Rheumatic Drug therapy in a subject; wherein the kit comprises at least one antibody against CD45RA and at least one antibody against FoxP3, wherein the kit further comprises at least one antibody selected from the group comprising: an antibody against CD4, an antibody against CD25, an antibody against CD127, and combinations thereof; wherein the kit optionally further comprises instructions for use.

Optionally, there is provided use of a kit for predicting responsiveness to a Disease Modifying Anti-Rheumatic Drug therapy in a subject; wherein the kit comprises at least one labelled antibody against CD45RA and at least one labelled antibody against FoxP3, wherein the kit further comprises at least one labelled antibody selected from the group comprising: a labelled antibody against CD4, a labelled antibody against CD25, a labelled antibody against CD127, and combinations thereof; wherein the kit optionally further comprises instructions for use.

Optionally, the kit comprises labelled antibodies. Optionally, the antibody against CD45RA is a labelled antibody against CD45RA. Optionally, the antibody against FoxP3 is a labelled antibody against FoxP3. Optionally, the antibody against CD4 is a labelled antibody against CD4. Optionally, the antibody against CD25 is a labelled antibody against CD25. Optionally, the antibody against CD127 is a labelled antibody against CD127.

Optionally, there is provided a composition for use in a method of diagnosis of responsiveness to Disease Modifying Anti-Rheumatic Drug therapy in a subject; wherein the composition comprises at least one antibody against CD45RA, and at least one antibody against FoxP3.

Optionally, there is provided a composition for use in a method of diagnosis of predicted responsiveness to Disease Modifying Anti-Rheumatic Drug therapy in a subject; wherein the composition comprises at least one antibody against CD45RA, and at least one antibody against FoxP3.

Optionally, there is provided a composition for use in a method of diagnosis in vivo of responsiveness to Disease Modifying Anti-Rheumatic Drug therapy in a subject; wherein the composition comprises at least one antibody against CD45RA, and at least one antibody against FoxP3.

Optionally, there is provided a composition for use in a method of diagnosis in vivo of predicted responsiveness to Disease Modifying Anti-Rheumatic Drug therapy in a subject; wherein the composition comprises at least one antibody against CD45RA, and at least one antibody against FoxP3.

Optionally, the composition further comprises at least one antibody selected from the group comprising: an antibody against CD4, an antibody against CD25, an antibody against CD127, and combinations thereof; wherein the kit optionally further comprises instructions for use.

Optionally, the composition comprises labelled antibodies. Optionally, the antibody against CD45RA is a labelled antibody against CD45RA. Optionally, the antibody against FoxP3 is a labelled antibody against FoxP3. Optionally, the antibody against CD4 is a labelled antibody against CD4. Optionally, the antibody against CD25 is a labelled antibody against CD25. Optionally, the antibody against CD127 is a labelled antibody against CD127.

Optionally, the composition comprises the kit of the second aspect of the present invention.

### BRIEF DESCRIPTION OF THE DRAWINGS

FIG. 1

(A) FACS dot plot showing gating of (i) lymphocyte population and subsequent CD3⁺CD45⁺CD4⁺CD25⁺CD127⁻ Tregs ((ii)-(iv)). (B) Chart representing the relative number of CD4⁺CD25⁺CD127⁻ Tregs in healthy controls and RA patients. P values shown were obtained using Mann Whitney tests. Central bar represents median value, error bars represent interquartile range. HC=healthy controls (n=21), DN=DMARD naïve (n=6), DR=DMARD responders (n=24) and DNR=DMARD non-responders (n=31).

FIG. 2

Charts representing (A) proportion of naïve CD45RA⁺FoxP3⁻ cells relative to the total number of circulating Tregs in RA patients and healthy controls, (B) proportion of naïve CD45RA⁺FoxP3⁺ cells relative to the total number of circulating Tregs in RA patients and healthy controls, (C) proportion of memory CD45RA⁻FoxP3⁺ cells relative to the total number of circulating Tregs in RA patients and healthy controls. P values shown were obtained using Mann Whitney tests. Central bar represents median value, error bars represent interquartile range. HC=healthy controls (n=13), DN=DMARD naïve (n=6), DR=DMARD responder (n=24), D-NR=DMARD non-responder (n=31).

FIG. 3

Charts representing (A) the absolute number of monocytes as measured by FBC in healthy controls and RA patients; (B) the percentage of classical monocytes in healthy controls and RA patients; (C) the percentage of non-classical monocytes in healthy controls and RA patients; and (D) the percentage of intermediate monocytes in healthy controls and RA patients. P values shown were obtained using unpaired t-tests or Mann Whitney tests depending on normality of distribution. Central bar represents median value, error bars represent interquartile range. HC=healthy controls, DN=DMARD naïve, DR=DMARD responder, D-NR=DMARD non-responder.

FIG. 4

Charts representing (A) the relative number of CD169⁺ classical monocytes in healthy controls and RA patients, (B) the relative number of non-classical monocytes in healthy controls and RA patients and (C) the relative number of intermediate monocytes in healthy controls and RA patients. P values shown were obtained using Mann Whitney tests. Central bar represents median value, error bars represent interquartile range. HC=healthy controls (n=19), DN=DMARD naïve (n=6), DR=DMARD responder (n=24), D-NR=DMARD non-responder (n=32).

FIG. 5

(A) The relative number of CD43⁺ Tregs in healthy controls and RA patients. (B) A log scale graph of CD43 MFI in Tregs of healthy controls and RA patients. P values shown were obtained using Mann Whitney tests. Central bar represents median value, error bars represent interquartile range. HC=healthy controls, DN=DMARD naïve, DR=DMARD responder, D-NR=DMARD non-responder, MFI=Median fluorescence intensity.

FIG. 6

Charts representing (A) the association between the relative number of CD169⁺ classical monocytes and DAS28-ESR, (B) the association between the relative number of CD169⁺ non-classical monocytes and DAS28-ESR, and (C) the association between the relative number of CD169⁺ intermediate monocytes and DAS28-ESR. (D) The association between CD43⁺ Tregs and DAS28-ESR. Linear regression was used to assess significance, with 95% CI. Data points were included if DAS28-ESR was available and patients has a moderate or high disease activity, as defined by EULAR criteria.

FIG. 7

Charts representing flow cytometry data showing the change in relative numbers of FoxP3⁺ or NFκB⁺ Tregs following stimulation with PMA and/or Sia; specifically (A) FoxP3⁺CD43⁺ Treg cells, (B) FoxP3⁺CD43⁻ Treg cells, (C) NFκB⁺CD43⁺ Treg cells, and (D) NFκB⁺CD43⁻ Treg cells. Number of cell culture plate wells at each time point=2 (mean plotted), where each was labelled for FACS analysis. CTL-Control, PMA-Phorbol 12-myristate 13-acetate, Sia=Sialic acid.

FIG. 8

Charts representing ELISA data assessing secreted cytokine levels from cell culture supernatants of Tregs exposed to a range of conditions. (A) TNFα, (B) IL-10 and (C) IFNγ levels were assessed at time points Day 0, 1 and 2; and in a repeat experiment (D) TNFα, (E) IL-10 and (F) IFNγ levels were assessed at Day 0 and 1. Number of cell culture plate wells at each time point=2, where each sample was run in duplicate wells of the ELISA plate (mean plotted). CTL-Control, PMA-Phorbol 12-myristate 13-acetate, Sia=Sialic acid.

FIG. 9

(A) CD14⁺ cells were gated, where the positioning was based on negative controls. (B) A subsequent dot plot was used to subdivide monocyte subsets into CD14⁺ CD16⁻ classical (B, Q1), CD14⁺⁺CD16⁺intermediate (B, Q2) and CD14⁺CD16⁺⁺ non-classical (B, Q4). (C) A subsequent dot plot of (A) and (B) was used to analyse CD169 positivity (C Q2-2).

FIG. 10

(A) The relative number of CD43⁺ Tregs (Q2-1) was reduced after being placed in culture for 14 days in comparison to PBMCs analysed on the day of sampling. (B) (i) CTL cells without stimulation, (ii) The percentage of CD4 positivity (Q2) was reduced following PMA stimulation, compared to (i) CTL cells without stimulation.

### EXAMPLES

### Methods

### Study Design and Participants

Informed consent to participate was obtained from all patients and healthy control subjects. Diagnosis for all patients fulfilled the 2010 American College of Rheumatology (ACR) criteria for Rheumatoid Arthritis. Patients were treated with one or more conventional disease-modifying anti-rheumatic drugs (DMARDs), including MTX, sulfasalazine, leflunomide and hydroxychloroquine. Patients were classified as ‘responders’ or ‘non-responders’ according to the National Institute for Health and Clinical Excellence (NICE) guidelines, where responders exhibit a change in DAS28-ESR of >1.2 following treatment. Blood samples and retrospective clinical data were collected at the time of consent (Table 1). Healthy control subjects were also recruited to the study, excluding individuals with any chronic inflammatory or autoimmune disorder. Office for Research Ethics Committees Northern Ireland (ORECNI) and Ulster University Research Ethics Committee (UREC) approvals were obtained for the study.

TABLE 1
Demographics of patients treated with conventional disease-modifying
anti-rheumatic drugs, taken from clinic notes of the day of sampling.

| | HC | | DN | | DR | | DNR | |
|---|---|---|---|---|---|---|---|---|
| | (n = 21) | | (n = 6) | | (n = 24) | | (n = 32) | |
| Female, n (%) | 16 | (76.2) | 5 | (83.3) | 22 | (91.7) | 27 | (81.8) |
| Age, mean (SD), years | 35.9 | (9.2) | 42.5 | (16.7) | 59.7 | (12.2) | 57.7 | (10.1) |

| | | | | | | |
|---|---|---|---|---|---|---|
| Disease duration, mean (SD), years | N/A | N/A | 5.0 | (3.8) | 10.8 | (12.1) |

| | | | | |
|---|---|---|---|---|
| Full Blood Count | | | | |
| Lymphocytes, mean (SD), 10⁹/L | 1.9 (0.4), | 1.6 (0.5), | 1.7 (0.3), | 1.6 (0.5), |
| | n = 19 | n = 6 | n = 24 | n = 26 |
| Monocytes, mean (SD), 10⁹/L | 0.5 (0.3), | 0.6 (0.2), | 0.5 (0.1), | 0.6 (0.3), |
| | n = 19 | n = 6 | n = 24 | n = 26 |
| Erythrocyte Sedimentation Rate | N/A | 23.5, (15.2), | 12.2 (7.9), | 24.1 (20.0), |
| (ESR), mean (SD), mm/hr | | n = 6 | n = 24 | n = 29 |
| C-reactive Protein (CRP), mean (SD), | N/A | 6.1 (2.9), | 5.3 (7.3), | 10.6 (9.4), |
| mg/L | | n = 6 | n = 22 | n = 28 |
| Disease Activity Score in 28 Joints | N/A | 4.3 (0.4), | 2.6 (1.0), | 4.9 (1.1), |
| (DAS28-ESR), mean (SD) | | n = 4 | n = 24 | n = 19 |

HC = healthy control, DN = DMARD naïve, DR = DMARD responder, DNR = DMARD non-responder.

### Blood Processing

Blood samples collected for peripheral blood mononuclear cell (PBMC) isolation were collected in tripotassium ethylenediaminetetraacetic acid (K₃EDTA) coated tubes (Aquilant Scientific, UK). Phlebotomy was performed by a research nurse or by a qualified member of the research team. Blood tubes were stored at room temperature (about 18° C. to about 23° C.) until processing. Histopaque-1077 (Sigma-Aldrich®, UK) density-gradient buffer was used to isolate PBMCs from blood samples.

### Cell Immunophenotyping

PBMCs were labelled with antibodies (Becton Dickinson®, UK) against CD3, CD4, CD25, CD45, CD45RA, CD127 and CD43 antigens according to the manufacturer's instructions to assess relative Treg numbers. When FoxP3 and nuclear factor kappa-light-chain-enhancer of activated B cells (NFκB) antibody labelling was required, cells were pretreated with a lymphocyte membrane permeabilising solution (BD FACS™ Permeabilizing solution 2; Becton Dickinson®, UK). For monocyte analysis, cells were firstly enriched using a pan monocyte isolation kit and benchtop magnetic cell sorting device (autoMACS Pro separator; Miltenyi Biotec®). Enriched monocytes were labelled with antibodies (Becton Dickinson®, UK) against CD14, CD16 and CD169 antigens. Cell populations were analysed using fluorescence-activated cell sorting (FACS) in a flow cytometer with sorting capability (FACSAria™ III; Becton Dickinson, UK) with appropriate flow cytometer software (BD FACSDiva™ software version 8.0.1; Becton Dickinson, UK). Negative controls including unlabelled cells and isotype matched control antibodies were used to determine gating strategies. The positivity of a specific cell surface marker in labelled samples compared to the negative control was analysed as a percentage of the parent population. The median fluorescence intensity (MFI), indicating cell surface density of a specific marker, was also noted for cell populations. Mann Whitney tests were used to assess the statistical significance of differences between sample groups and graphs depict median values with error bars representing the interquartile range.

### Cell Expansion and Culture

CD4⁺CD25⁺CD127⁻ Treg cells were sorted by FACS into phosphate buffered saline (PBS) containing 20% AB serum (Sigma-Aldrich®, UK), then washed and resuspended in serum-free cell culture medium (TexsMACS™ medium; Miltenyi Biotec®, UK), supplemented with 5% AB serum (Sigma-Aldrich®, UK), 1% Penicillin-Streptomycin (Gibco®, Ireland) and human IL-2 (Miltenyi Biotec®, UK). Trypan blue (Sigma-Aldrich®, UK) was used to ensure cell viability after sorting was >90-95%. A human Treg expansion kit (Miltenyi Biotec, UK) was used to induce Treg proliferation in order to expand the population to sufficient cell numbers for experiments. Following 14 days of expansion, Tregs were stimulated with 10 ng/ml phorbol 12-myristate 13-acetate (PMA) (Sigma-Aldrich®, UK) and 500 ng/ml ionomycin (10) (Invitrogen®) for 24 hours. During the same 24-hour stimulation, Tregs were incubated with or without 10 mM sialic acid (Sia) (Sigma-Aldrich®, UK). At baseline, 24 hr and 48 hr time points (Day 0, Day 1 and Day 2), cells were removed from culture and labelled prior to FACS analysis of extracellular markers CD4, CD25, CD127 and CD43, and intracellular markers NFκB and FoxP3. Cell culture conditioned media was retained and analysed for secreted cytokine levels by enzyme-linked immunosorbent assay (ELISA), described below. A preliminary experiment included a Day 2 time-point, however latter experiments were concluded at the Day 1 time-point as this was sufficient to observe Sia treatment effects. Statistical analysis was carried out using analysis and graphing software (Prism version 5.01; GraphPad), where Mann Whitney tests were used to assess the statistical significance of differences between experimental groups.

### Enzyme-Linked Immunosorbent Assay (ELISA)

Following cell culture experiments, conditioned media were analysed to quantify TNFα, IL-10 and IFN-γ by ELISA using appropriate ELISA reagent kits (DuoSet® ELISA development kits; R&D Systems®, UK) according to the manufacturer's instructions. The optical density (OD) was determined using a spectrophotometer (Epoch™ microplate spectrophotometer; BioTek® Ltd, UK) with appropriate software (Gen5™; BioTek® Ltd, UK). The plate was read at a wavelength of 450 nm and wavelength correction of OD at 540 nm used. The cytokine concentrations within the conditioned media samples were then interpolated from the construct standard curve.

### Results

### Study Participant Demographics

Participant demographics were calculated for DMARD responder and non-responder subgroups, as median±standard deviation (SD) (Table 2). Statistical analysis was performed, comparing clinical data initially between DMARD responders and non-responders. The median disease duration and the median DAS28-ESR scores were significantly higher in non-responder patients (9.00 years; 5.51 DAS28) compared to responders (4.50 years; 2.34 DAS28) (p<0.01). Median ESR was also significantly increased in non-responders (21.5 mm/hr) compared to responders (11.0 mm/hr) (p=0.01), however there was no significant difference between CRP levels of each group (p=0.08). Demographic information was not compared between RA patients and healthy control subjects because clinical data was not recorded for the latter. Also, due to the small number of DMARD naïve patients (n=6), comparisons were not made using this study group.

TABLE 2
Statistical differences between clinical data
of DMARD responders vs non-responders.

| | DR | DNR | | |
|---|---|---|---|---|
| | (Median ± SD) | | p value | |
| Disease Duration | 4.50 ± 3.85 | 9.00 ± 11.43 | <0.01 | (***) |
| DAS28-ESR | 2.34 ± 1.05 | 5.51 ± 1.39 | <0.01 | (***) |
| ESR | 11.00 ± 7.93 | 21.50 ± 16.10 | 0.01 | (*) |
| CRP | 2.75 ± 7.30 | 8.80 ± 24.80 | 0.08 | (ns) |

DR = DMARD responders, DNR = DMARD non-responder
P values shown were obtained using Mann Whitney tests. Error bars represent interquartile range.

### Relative Numbers of Circulating Tregs in RA

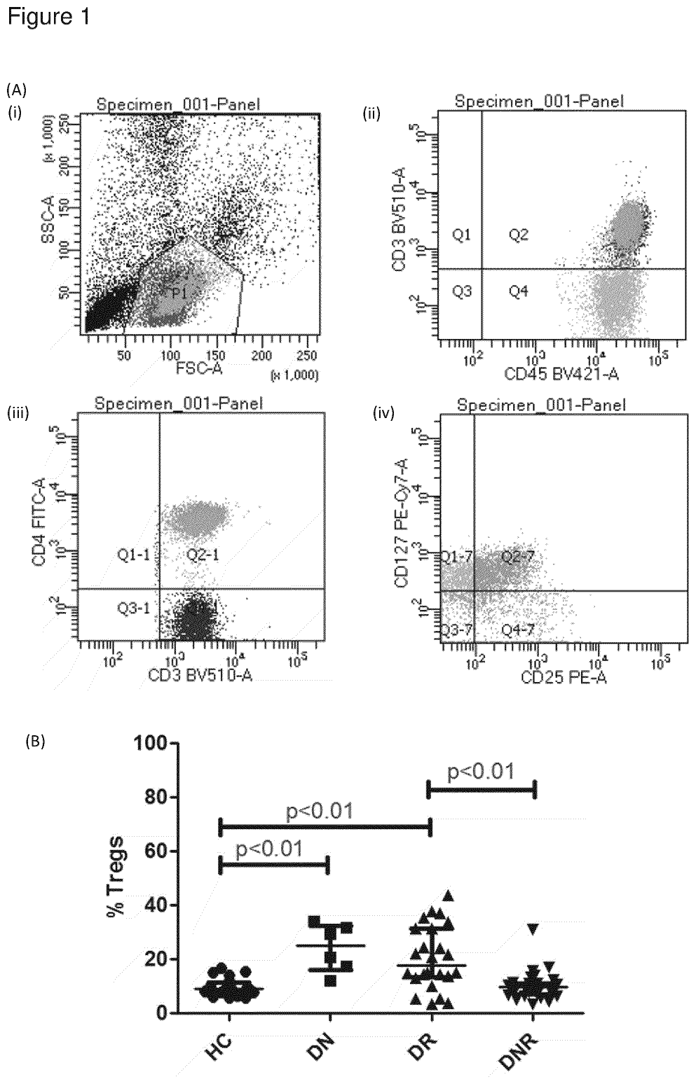

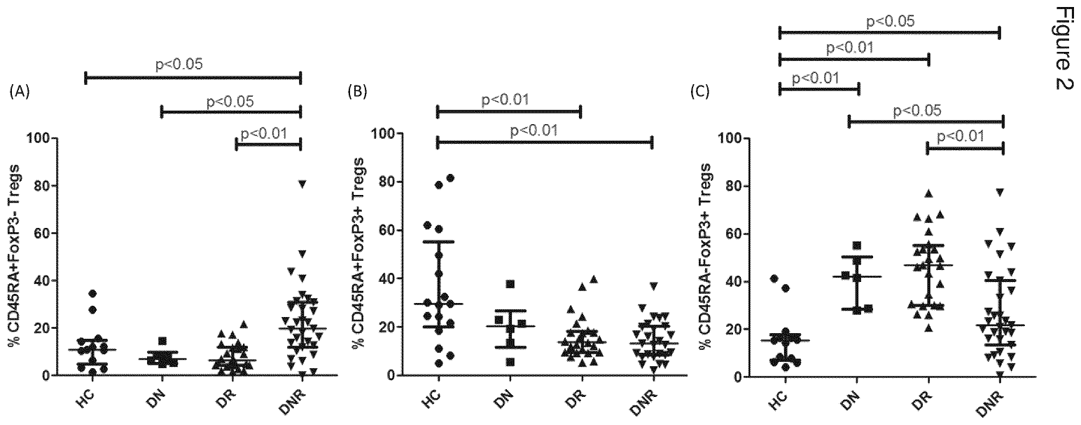

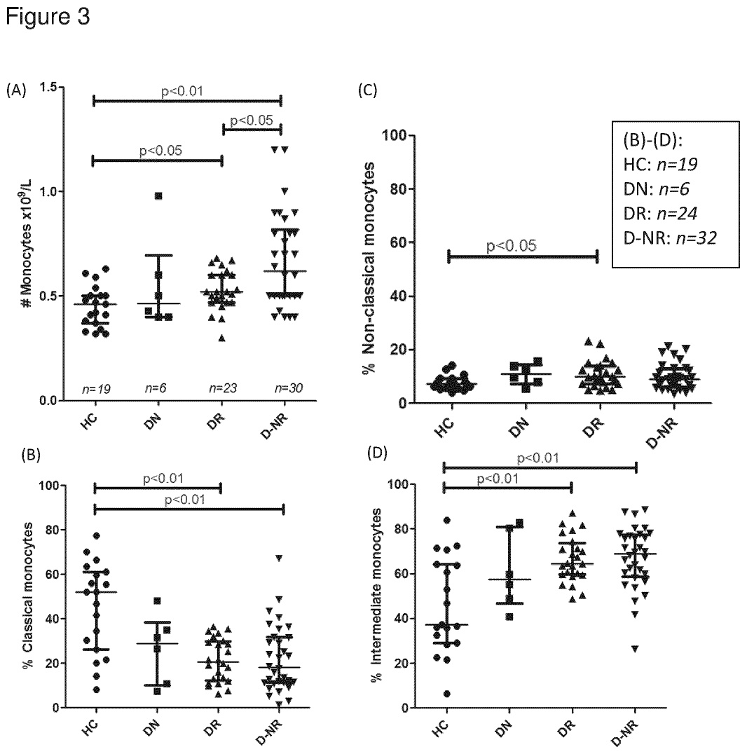

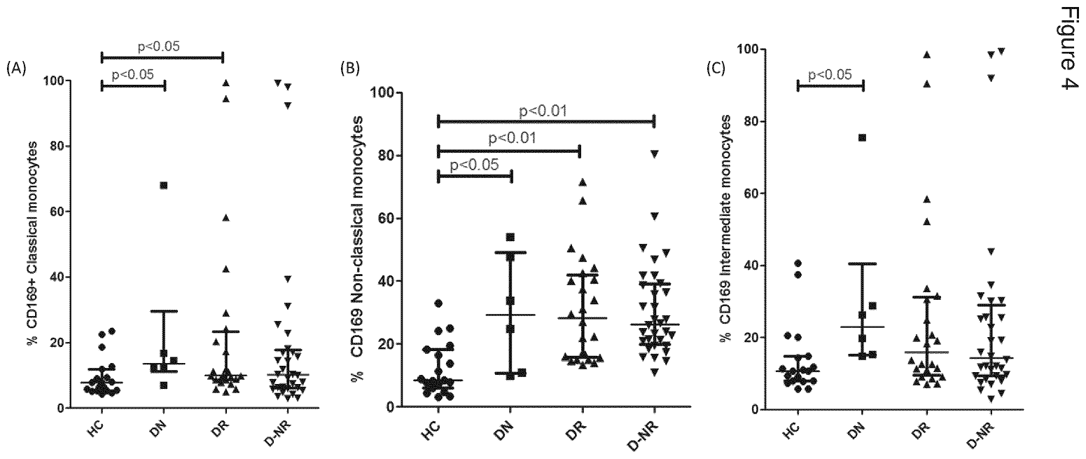

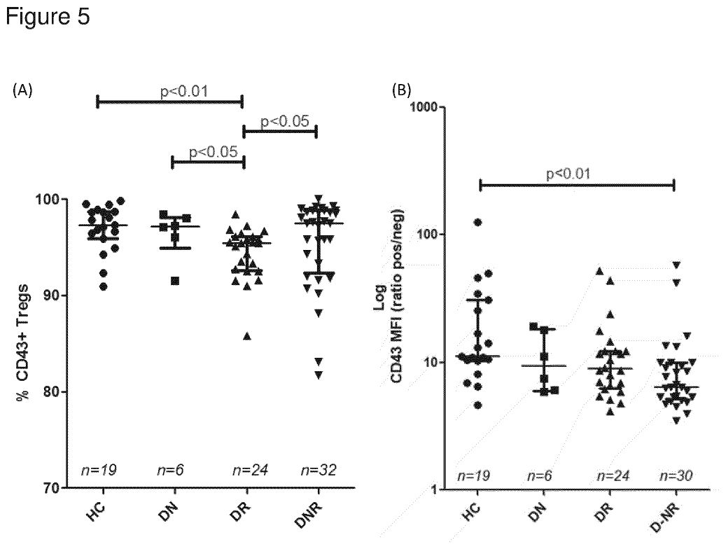

Relative CD4⁺CD25⁺CD127⁻ Treg numbers from healthy subjects and RA patient subgroups were calculated as a percentage of CD3⁺CD45⁺ T cells (FIG. 1). Circulating relative Treg numbers were significantly increased in DMARD naïve (25.00±8.77, n=6, p<0.01) and DMARD responder RA patients (17.80±11.75, n=24, p<0.01) compared to healthy controls (9.10±3.21, n=21). Furthermore, RA patients who were unresponsive to DMARD treatment had a significantly lower percentage of circulating Tregs (9.50±4.98, n=31, p<0.01) compared to those who have responded.

### Treg Activation in RA (FoxP3 and CD45RA)

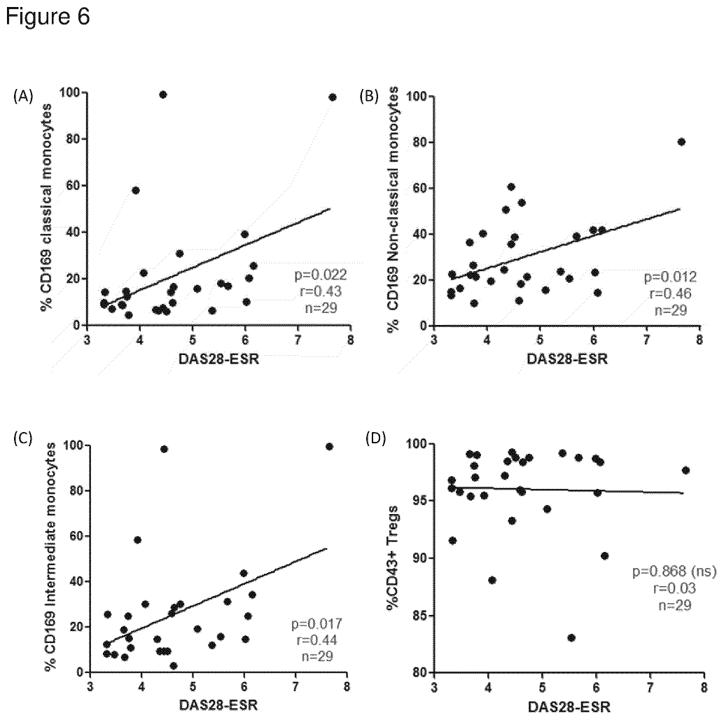

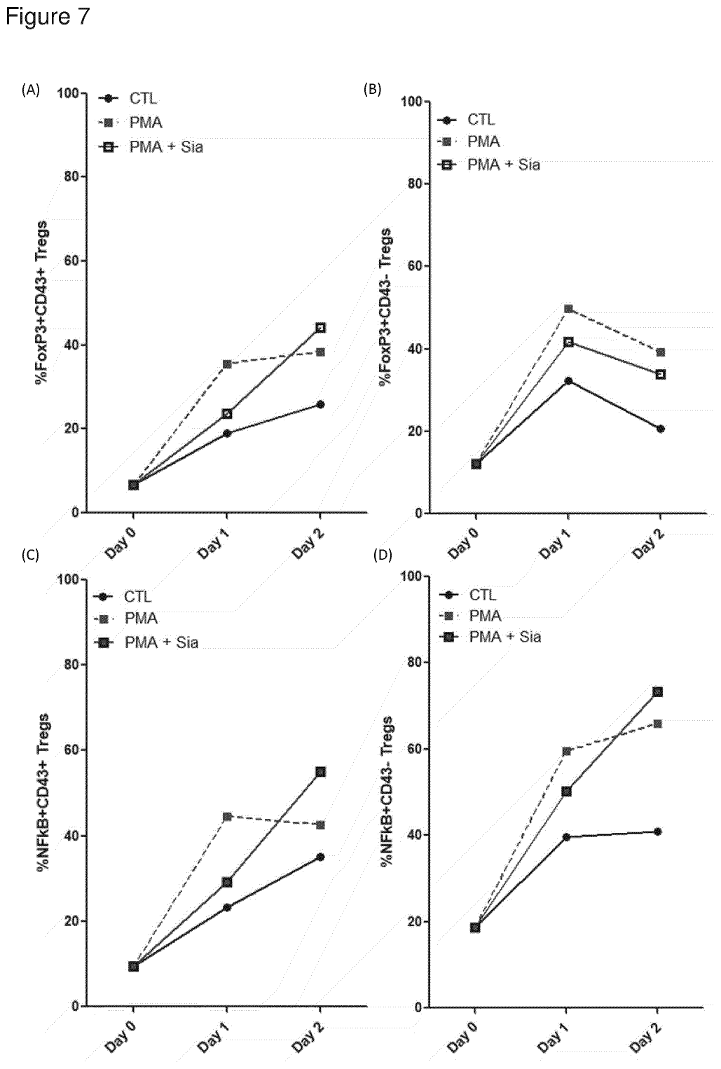

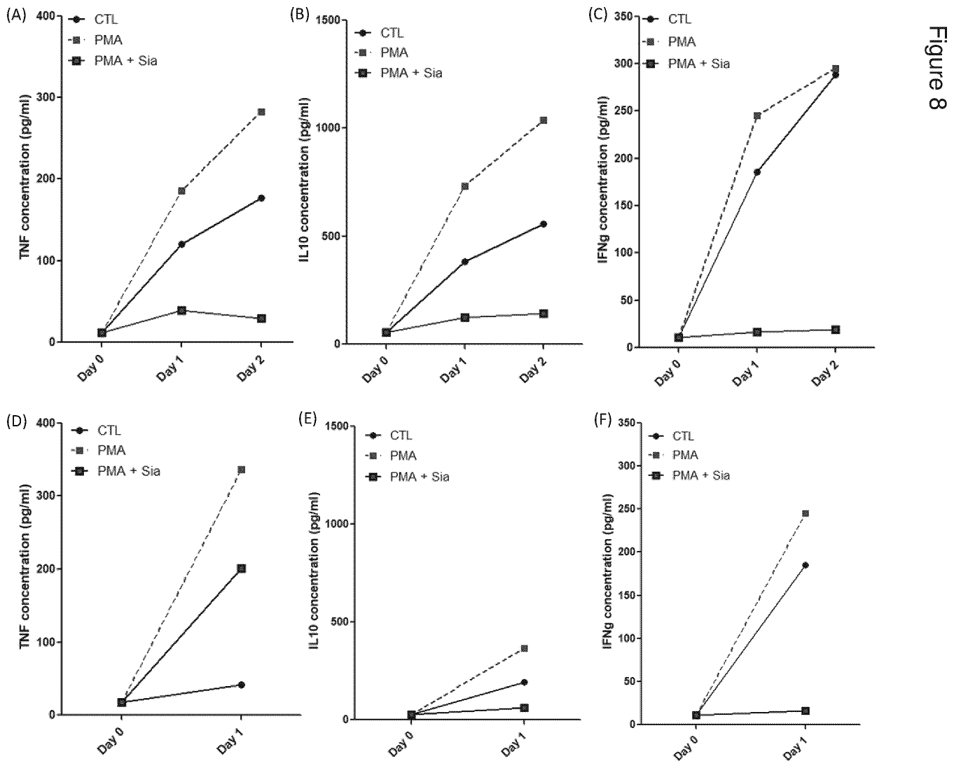

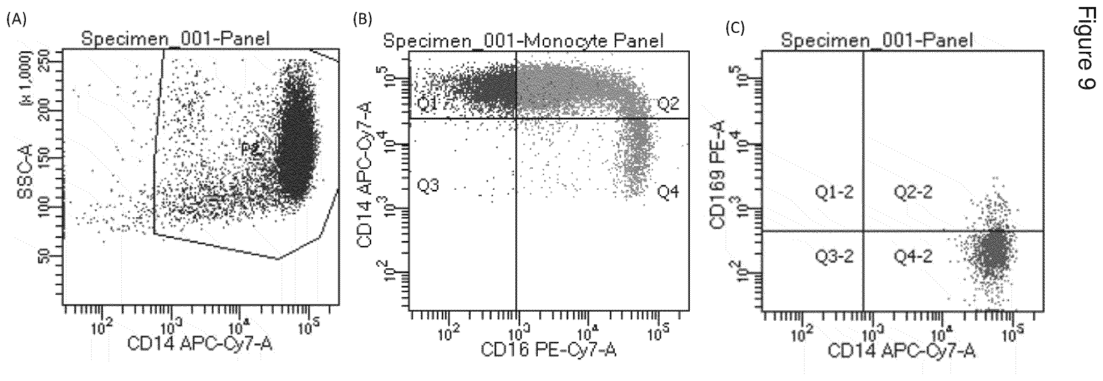

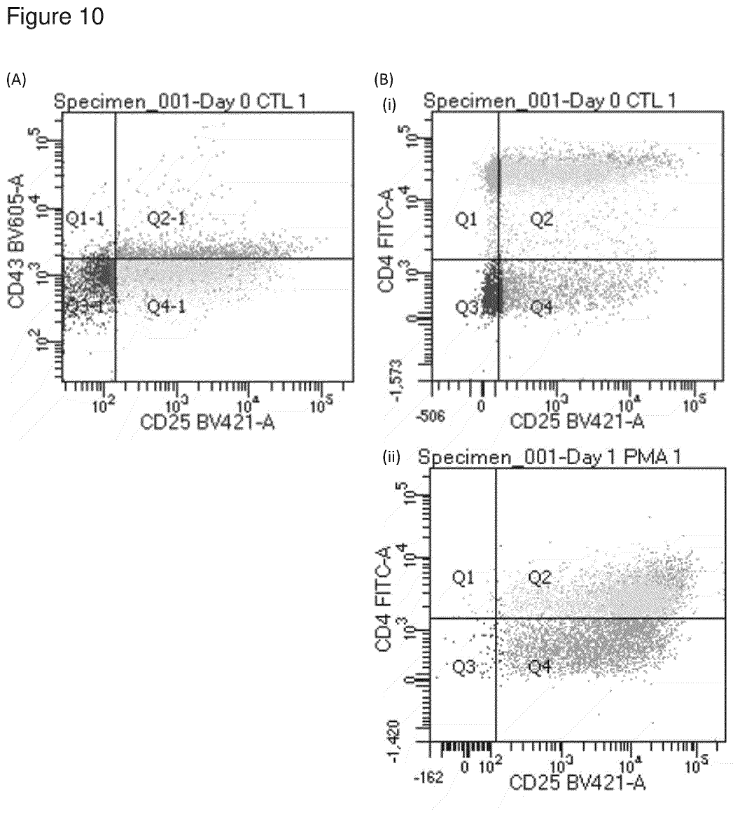

The relative numbers of CD45RA⁺ and activated FoxP3⁺ Tregs (CD4⁺CD25⁺CD127⁻) were analysed in healthy controls and RA patients (FIG. 2). The relative number of CD45RA⁺FoxP3⁻ Tregs was significantly higher in DMARD non-responders (19.90±16.39, n=31), compared to healthy controls (10.90±9.47, n=13, p<0.05), DMARD naïve (6.90±3.51, n=6, p<0.05) and DMARD responder patients (6.50±5.54, n=24, p<0.01) (FIG. 2A). Furthermore, the relative number of CD45RA⁺FoxP3⁺ Tregs was significantly reduced in DMARD non-responders (13.20±8.33, n=27) and DMARD responders (13.60±8.81, n=24, p<0.01) compared to healthy controls (29.60±23.27, n=13, p<0.01) (FIG. 2B). The relative number of CD45RA⁻FoxP3⁺ Tregs was significantly elevated in DMARD naïve (42.00±10.82, n=6, p<0.01), DMARD responder (46.70±15.42, n=24, p<0.01) and DMARD non-responder patients (21.60±18.66, n=31, p<0.05), in comparison to healthy controls (15.40±11.44, n=13) (FIG. 2C). However, DMARD non-responders exhibited a significantly lower relative number of CD45RA⁻FoxP3⁺ Tregs compared to DMARD naïve (p<0.05) and DMARD responder patients (p<0.01).

### Monocytes in Healthy and RA Subgroups

The absolute number of circulating monocytes in DMARD responders (0.527×10⁹/l±0.097, n=23) and non-responders (0.670×10⁹/l±0.229, n=30) were significantly higher relative to healthy controls (0.452×10⁹/l±0.097, n=19, p=0.019 and 0.0003 respectively) (FIG. 3A). There was no statistically significant difference between healthy controls and DMARD naïve patients (0.552×10⁹/l±0.223, n=6, p=0.407). Additionally, the absolute number of monocytes in responders was significantly lower than non-responders (p=0.041).

Monocyte subsets were assessed as shown in FIG. 9. The relative number of CD14⁺⁺CD16⁻ classical monocytes was significantly lower in RA responders (21.37%±9.68, n=24) and non-responders (22.09%±15.03, n=32) to DMARD treatment, compared to healthy controls (45.03%±20.33, n=19, p<0.001) (FIG. 3B). However, the relative number of CD14⁺CD16⁺⁺ non-classical monocytes was increased in RA patients compared to healthy controls (7.595%±2.606, n=19), the difference statistically significant in DMARD responders (10.93%±4.978, n=24, p=0.017) (FIG. 3C). Furthermore, the relative number of CD14⁺⁺CD16⁻ intermediate monocytes was significantly increased in both responders (66.37%±10.31, n=24, p=0.004) and non-responders (66.90%±13.90, n=32, p=0.001) compared to healthy controls (45.83%±21.31, n=19) (FIG. 3D). However, no statistically significant difference was observed between responders and non-responders across any of the monocyte subsets.

### CD169 (Siglec-1) Expression on Monocyte Subsets in RA and Healthy

Overall, the percentage of CD169 positive monocytes was elevated in RA patient subgroups compared to healthy controls (FIG. 4). DMARD naïve RA patients (21.83%±22.80, n=6, p=0.039) and DMARD responders (21.89%±26.31, n=24, p=0.014) have a significantly increased relative number of CD169, classical monocytes, compared to healthy controls (9.426%±5.890, n=19), however the difference was not statistically significant in DMARD non-responders (19.83%±26.40, n=32, p=0.185) compared to healthy controls (FIG. 4A). A similar observation was made in non-classical monocytes, where CD169 percentage was increased in DMARD naïve (30.10%±18.41, 0.014), DMARD responder (30.95%±16.73, p<0.001) and DMARD non-responder (30.68%±15.05, p<0.001) RA patients compared to healthy controls (12.15%±8.392) (FIG. 4B). Additionally, CD169 percentage was increased in intermediate monocytes for each patient group compared to healthy controls (13.87%±9.778), however only with statistical significance in DMARD naïve patients (30.02%±23.00, p=0.011) (FIG. 4C). Furthermore, the MFI of CD169 on the surface of classical and intermediate monocytes was not significantly increased in DMARD non-responders compared to other patient groups and healthy controls.

The Relationships Between CD169⁺ Monocytes or CD43⁺ Tregs and Disease Activity

The relative number of CD43⁺ Tregs was reduced in each patient group compared to healthy controls (96.94%±2.434, n=19), however only DMARD responders display a statistically significant difference (94.37%±2.698, n=24, p=0.001) (FIG. 5). Furthermore, the relative number of CD43⁺ Tregs was significantly reduced in DMARD responders compared to DMARD naïve (96.37%±2.524, n=6, p=0.049) and non-responder patients (95.41%±4.647, n=32, p=0.024). Additionally, the MFI of CD43 on the surface of Tregs was reduced in each RA patient group compared to healthy controls (23.44 MFI±27.96), however a statistically significant difference was only observed in non-responders (10.34 MFI±11.55, p=<0.001).

A statistically significant positive association was observed between DAS28-ESR and the percentage of CD169 classical (p=0.022, r=0.424, n=29), non-classical (p=0.012, r=0.459, n=29) and intermediate (p=0.017, r=0.440, n=29) monocytes (FIG. 6A-C). However, the relative number of CD169⁺ non-classical monocytes had the most significant relationship with DAS28-ESR versus other subgroups. The relative number of CD43⁺ Tregs had no significant association with DAS28-ESR (p=0.868, r=0.032, n=29) (FIG. 6D).

The Effect of Sialic Acid on In Vitro Tregs Simulated with PMA

After stimulation with PMA, a reduction in CD4 cell-surface expression was observed (FIG. 10). Additionally, the percentage of CD43⁺ Tregs was significantly lower following in vitro expansion, compared to that observed from fresh PBMCs analysed on the day of collection. The percentages of NFκB and FoxP3 expressing cells were assessed in CD43⁺ (FIGS. 7A and 7C) and CD43⁻ Tregs (FIGS. 7B and 7D). Stimulation with PMA increased the percentage of cells with intracellular expression of NFκB (p<0.05) and FoxP3 (p<0.01) in both CD43⁺ and CD43⁻ Tregs at each time point. Addition of Sia reduced the relative numbers of NFκB⁺ on Day 1 (not statistically significant) and FoxP3⁺ Tregs on Day 1 and Day 2, whereby the latter displayed a statistically significant reduction over time (p<0.05). The same effect was observed after 2 days in CD43⁻FoxP3⁺ Tregs but not in CD43⁺FoxP3⁺ Tregs. Furthermore, after 2 days, the addition of Sia had no significant effect on NFκB Tregs.

Concentrations of cytokines secreted into the surrounding media including TNFα, IL-10 and IFNγ were analysed by ELISA (FIG. 8A-8C). PMA significantly increased levels of TNFα (p<0.05) and IL-10 (p<0.05) on both Day 1 and Day 2, relative to controls. IFNγ levels were also significantly higher with PMA stimulation relative to controls on Day 1 (p<0.01), but not on Day 2. At each time point, addition of Sia caused a significant reduction TNFα, IL-10 and IFNγ (p<0.01).

### DISCUSSION

This study provides insight into how number and phenotype of peripheral Tregs relate to disease activity after DMARD treatment response in RA patients. Furthermore, novel findings supporting a potential role for monocyte involvement in modulation of Treg activity are presented, with particular focus on the interaction between CD169⁺ monocytes and CD43⁺ Tregs.

This study indicates DMARD naïve and responder patients have an increased relative number of Tregs compared to healthy controls, however DMARD non-responders exhibit reduced Treg numbers compared to responders. This is in agreement with prior work, whereby patients who responded to anti-TNF treatment had increased relative numbers of circulating Tregs compared to their own baseline samples, suggesting the downregulation of Tregs in RA may be reversed as a consequence of treatment response. This is one explanation for the significant reduction of Tregs in DMARD non-responders observed in the current study, compared to responders. However, the current study did not indicate a statistically significant difference in relative Treg numbers between DMARD naïve patients and DMARD responders. This may in part be due to the modest number of patients in the DMARD naïve group (n=6) compared to responders (n=24), thus making statistical comparison difficult.

CD45RA⁺ Tregs were also analysed, representing a naïve subset of Tregs that were not activated by prior antigen encounter. Within the CD45RA⁺ Tregs, the mean percentage of FoxP3⁻ cells was highest in DMARD non-responders compared to all of the other study groups. Furthermore, the relative number of FoxP3⁺ cells was reduced in DMARD responders and non-responders compared to healthy controls. This suggests the activation of naïve Tregs is reduced in RA patients, in agreement with previous findings. Interestingly, the relative number of CD45RA⁻FoxP3⁺ Tregs was elevated in RA patients compared to healthy controls, in agreement with a recent study. This data suggests there is increased activation of CD45RA⁻ memory Tregs in RA peripheral blood, relative to healthy subjects. However, a significantly lower relative number of CD45RA⁻FoxP3⁺ Tregs is observed in DMARD non-responders compared to DMARD naïve and DMARD responder patients. These findings suggest DMARD non-responder RA patients exhibit reduced numbers of circulating active naïve and memory Tregs, compared to DMARD responder RA patients.

Increased absolute monocyte numbers were observed from full blood count (FBC) results of RA patients compared to healthy controls, which confirms that inflammatory recruitment of circulating monocytes can be detected. Absolute numbers of monocytes were significantly reduced in DMARD responders compared to non-responders. A recent report also describes how circulating monocytes increase in number during active disease. This explains why in the current study, monocytes are found in higher numbers in RA patients that remain unresponsive to DMARDs, who have significantly higher disease activity.

In concordance with previous studies, the relative number of CD16⁺ monocytes was observed to be increased in RA, whereas CD16⁻ classical monocytes were reduced. A previous study also found an increase in CD16⁺ intermediate monocytes in patients with longer disease duration compared to those with less than 2 years duration, however similar changes in CD16⁺ non-classical monocytes were not noted. This increase in CD16+ intermediate monocytes was not observed in the current study, possibly due to the low number of DMARD naïve patients. However, a similar pattern was observed whereby DMARD responders and non-responders had higher intermediate monocytes compared to healthy controls. Additionally, intermediate monocytes, which could represent a middle state between classical and non-classical, adopted a similar pattern to non-classical monocytes with an increased percentage noted in RA compared to healthy controls. This indicates that CD16⁺ monocytes may have more impact on RA pathogenesis than previously considered.

CD16⁺ monocytes are more likely to differentiate into dendritic cells, which are believed to play a pivotal role in RA progression and have been suggested as a potential target for therapy. However, despite the recent finding that classical and intermediate monocytes had predictive value in determining treatment response, the data presented in the current study demonstrates no significant difference between responders and non-responders for any of the monocyte subsets. This may be due to modest patient numbers in the current study.

Despite the differences observed in the relative number of each monocyte subset, the relative number of CD169 positive classical, non-classical and intermediate monocytes was increased in RA compared to healthy controls. A novel finding of the current study is that the relative number of CD169⁺ non-classical monocytes were found at significantly increased levels in RA compared to healthy control. This result further suggests CD16⁺ monocytes may have a significant role in RA pathogenesis compared to CD16⁻ monocytes, along with CD169 contributing to disease progression.

The cell surface density (MFI) of CD43 on circulating Tregs, as well as the relative number of CD43⁺ Tregs, was found to be lower in RA compared to healthy control. This has not previously been reported and therefore represents a novel finding. There is limited knowledge of CD43 as a Treg transmembrane sialoglycoprotein, however previous studies have demonstrated its involvement in T cell activation and proliferation. Therefore, the reduction in CD43 density and percentage observed in this study may be related to the reduced activation observed in Tregs. However, contrary to this theory, DMARD responders have significantly lower relative numbers of CD43⁺ Tregs compared to non-responders. Conversely, although no significant difference was observed, the MFI of CD43 is increased in responders compared to non-responders. This means that although there may be lower CD43 as a percentage of Tregs in responders compared to non-responders, there is increased CD43 cell surface density per CD43⁺ Treg cell in responders. Therefore, the increase in CD43 per cell in responders may be related to increased T cell activation, as previously suggested, however further investigation is needed to confirm this. Although the CD43 profile may be difficult to interpret in this preliminary analysis, the difference in percentage between responders and non-responders would suggest CD43 has the potential as a surrogate marker of treatment response.

When monocyte subset was considered, the relative number of CD169⁺ monocytes had a significant association with DAS28-ESR. Interestingly, CD169⁺ non-classical monocytes had a strongest association with DAS28-ESR, whereas CD169⁺ classical monocytes had the weakest of the three subsets. This implies CD16⁺ monocytes are more closely associated with disease activity than CD16⁻ monocytes. There was no association between the relative number of CD43⁺ Tregs and DAS28-ESR, suggesting any impact of the CD169/CD43 relationship on disease activity is dominated by CD169.

The aim of the in vitro inflammatory model was to induce an activated state in Tregs stimulating their phenotype in RA, as measured by production of pro-inflammatory cytokines including TNFα and IL-6. Sia was added to the activated Tregs in order to mimic the effect of CD169, which can bind to and present this residue. CD169 binds to Sia in order to carry out its functions, including binding to ligands on the surface of Tregs. A previous study showed Tregs contain many Sia ligands on their cell surface. Although the purpose of the experiment was to determine cytokine and intracellular pathway responses, it cannot be definitively stated that CD169 has the same effect. Sia significantly reduced the activation of Tregs, as measured by decreased levels of secreted cytokines TNFα, IL-10 and IFNγ, as well as decreased relative numbers of NFκB⁺ and FoxP3⁺ Tregs. This agrees with previous findings, where Sia has been shown to reduce Treg activation as measured by a reduction of Treg suppressive function as well as activation markers including CD25. However, the impact on cytokine and transcription factors utilised in the current study has not previously been reported. This effect was only observed in NFκB⁺ Tregs only when they were activated with PMA/IO for 1 day. A similar effect was observed for NFκB⁺CD43⁺ and NFκB⁺CD43⁻ Tregs. However, after 2 days of stimulation Sia significantly reduced FoxP3⁺CD43⁻ Tregs, an effect which was not observed in FoxP3⁺CD43⁺ Tregs. The discrepancy between the relative number of NFκB+ and FoxP3+ Tregs, and cytokine levels on Day 2 of culture may occur as a result of an initial cytokine response within the first day of stimulation that is still detected in surrounding medium on Day 2.

Introducing Sia into PMA stimulated Treg cultures also significantly reduced TNFα, IL-10 and IFNγ cytokine secretion, an effect that was observed on both Day 1 and Day 2 of culture. The inventors postulate that Sia may mimic the action of CD169 on Tregs. It could be hypothesised that direct interaction of CD169 and CD43 could contribute to Treg modulation and ultimately disease activity in RA.

### CONCLUSION

The key findings of this study include the detection of activated Tregs in peripheral blood and their potential as surrogate markers which distinguish DMARD treatment response. This suggests reduced Treg activity may contribute to or be a consequence of treatment non-response.

This study also demonstrates the importance of considering CD16⁺ non-classical and intermediate monocytes as well as CD16⁻ classical monocytes in relation to RA, as these key cells also influence Treg response. CD16⁺ intermediate monocyte numbers suggest a greater impact of this cell subset on RA pathogenesis than previously considered. Furthermore, although CD169⁺ classical monocytes have previously been postulated to play a role in RA disease activity, we report a significant increase in CD169 expression of non-classical monocytes in all RA patient groups compared to healthy controls. Furthermore, this subset of cells has the most significant positive association with DAS28, suggesting it is perhaps a more important subset of monocytes for future study. This study has established that Sia suppresses Tregs in an in vitro model of inflammation, as measured by a reduction in inflammatory cytokine secretion, as well as a reduction in relative numbers of NFκB⁺ and FoxP3⁺ Tregs. We postulate this may mimic the action of CD169 on Tregs, however this could not be confirmed in the current study. From these results it could be hypothesised that direct interaction of CD169 and CD43 could contribute to Treg modulation and ultimately disease activity in RA, though further work is needed to confirm this.

In summary, this study presents novel evidence of the pivotal role of peripheral Tregs in RA disease activity, which may be driven by a specific subset of monocytes. Furthermore, this study suggests there is potential to assess disease activity by analysing circulating immune cells, which could enable earlier determination of treatment response.

The present invention could be used in at least two ways:
(a) To set up ex vivo (or in vitro) cultures of blood cells to test treatment combinations against, thus speeding up the process and reducing the amount of time the patient is exposed to ineffective treatment. Only when a drug combination produces the desired effect on the cell immunophenotype in the lab, would the treatment be used on the patient.
(b) Alternately a test for the identified cell immunophenotype could be used to monitor response to treatments on the fly with potential time saving advantage over current treatment regimes.

Either or both of the above uses could save clinician and patient time, reduce risk of severe outcomes and potentially lower overall costs on healthcare providers.

The terms healthy individual, healthy control subject, healthy control, healthy subject, and healthy are used interchangeably.

Cluster of differentiation (CD) nomenclature is used to identify several cell surface markers, including but not limited to: CD3, CD4, CD14, CD16, CD25, CD43, CD45, CD69, CD71, C127, CD169. For example, CD3 refers to cluster of differentiation 3.

Sialic acid-binding immunoglobulin-like lectin 1 (Siglec-1) is also known as CD169

Tregs can be identified by a CD4⁺CD25⁺CD127⁻ phenotype. Therefore, a CD45RA⁺FoxP3⁻ Treg can be identified by, and is synonymous with, a CD4⁺CD25⁺CD127⁻CD45RA⁺FoxP3⁻ phenotype; and a CD45RA⁻FoxP3⁺ Treg can be identified by, and is synonymous with, a CD4⁺CD25⁺CD127⁻CD45RA⁻ FoxP3⁺ phenotype.

T helper (Th) cells are also known as CD4⁺ cells or CD4⁺ T cells.

Several cytokines in the interleukin (IL) group are mentioned, including but not limited to: interleukin 2 (IL-2), interleukin 6 (IL-6), interleukin 10 (IL-10), interleukin 17 (IL-17), interleukin 23 (IL-23). For example, IL-2 refers to interleukin 2.

ESR refers to erythrocyte sedimentation rate. DAS28 refers to disease activity score of 28 joints. DAS28-ESR refers to disease activity score of 28 joints with erythrocyte sedimentation rate.

Th1 refers to Type 1 helper T cell. Th17 refers to T helper 17 cell.

FoxP3 refers to Forkhead box P3. CTLA-4 refers to cytotoxic T lymphocyte protein-4.

IFN-γ refers to interferon gamma.

TNF refers to tumor necrosis factor, while TNFα refers to tumor necrosis factor alpha.

1. An in vitro method for predicting responsiveness to a Disease Modifying Anti-Rheumatic Drug therapy in a subject, the method comprising the steps of:
a) providing a biological sample;
b) detecting the presence, absence, or quantitative level of a first marker in the biological sample, wherein the first marker is a regulatory T cell having a CD45RA⁺FoxP3⁻ phenotype;
c) correlating the presence, absence, or quantitative level of the first marker to the predicted responsiveness to the Disease Modifying Anti-Rheumatic Drug therapy in the subject.

2. The method of claim 1, the method comprising the steps of:
a) providing a biological sample;
b) detecting the presence, absence, or quantitative level of a first marker in the biological sample, wherein the first marker is a regulatory T cell having a CD45RA⁺FoxP3⁻ phenotype; and detecting the presence, absence, or quantitative level of a second marker in the biological sample, wherein the second marker is a regulatory T cell having a CD45RA⁻FoxP3⁺ phenotype;
c) correlating the presence, absence, or quantitative level of the first marker and the presence, absence, or quantitative level of the second marker; to the predicted responsiveness to the Disease Modifying Anti-Rheumatic Drug therapy in the subject.

3. The method of claim 1 or 2, wherein the detecting step (b) further comprises:
c) detecting the presence, absence or quantitative level of a third marker in the biological sample, wherein the third marker is a regulatory T cell.

4. The method of claim 3, wherein the third marker is a cell having a CD4⁺CD25⁺CD127⁻ phenotype.

5. The method of claim 3 or 4, wherein the quantitative level of the first marker is a relative level of the first marker as a proportion of the quantitative level of the third marker.

6. The method of any of claims 3 to 5, wherein the quantitative level of the second marker is a relative level of the second marker as a proportion of the quantitative level of the third marker.

7. The method of claim 5 or 6, wherein a relative level of the first marker greater than 20% correlates to a predicted low responsiveness to the Disease Modifying Anti-Rheumatic Drug therapy in the subject.

8. The method of any of claims 5 to 7, wherein a relative level of the first marker less than 20% correlates to a predicted high responsiveness to the Disease Modifying Anti-Rheumatic Drug therapy in the subject.

9. The method of any of claims 6 to 8, wherein a relative level of the second marker less than 26% correlates to a predicted low responsiveness to the Disease Modifying Anti-Rheumatic Drug therapy in the subject.

10. The method of any of claims 6 to 9, wherein a relative level of the second marker greater than 26% correlates to a predicted high responsiveness to the Disease Modifying Anti-Rheumatic Drug therapy in the subject.

11. The method of any preceding claim, wherein the biological sample substantially comprises a blood sample.

12. The method of any preceding claim, wherein the detecting step (b) comprises antibody labelling of at least one marker selected from the group comprising: the first marker, the second marker, the third marker, and combinations thereof.

13. The method of any preceding claim, wherein the detecting step (b) comprises flow cytometry, optionally fluorescence-activated cell sorting.

14. The method of any preceding claim, wherein the method further comprises a stimulating step
(a1), wherein providing step (a) precedes stimulating step (a1), and wherein stimulating step
(a1) precedes detecting step (b), wherein stimulating step (a1) comprises:
a1) stimulating the biological sample with the Disease Modifying Anti-Rheumatic Drug.

15. The method of claim 15 wherein the biological sample comprises a culture, optionally an in vitro culture.

16. A Disease Modifying Anti-Rheumatic Drug therapy response prediction kit, comprising at least one labelled antibody against CD45RA and at least one labelled antibody against FoxP3; optionally further comprising instructions for use.

17. The kit of claim 12, further comprising at least one labelled antibody selected from the group comprising: a labelled antibody against CD4, a labelled antibody against CD25, a labelled antibody against CD127, and combinations thereof.
